(19) United States
(12) Patent Application Publication
(10) Pub. No.: US 2026/0085353 A1
(43) Pub. Date: Mar. 26, 2026

(54) METHODS AND COMPOSITIONS FOR IN SITU SEQUENCING

(71) Applicant: 10x Genomics, Inc., Pleasanton, CA (US)

(72) Inventor: Trevor STOCKDALE, Walnut Creek, CA (US)

(21) Appl. No.: 19/334,723

(22) Filed: Sep. 19, 2025

Related U.S. Application Data
(60) Provisional application No. 63/697,263, filed on Sep. 20, 2024.

(51) Int. Cl. C12Q 1/6874; C12Q 1/6806

(52) U.S. Cl. CPC C12Q 1/6874; C12Q 1/6806

(57) ABSTRACT

The present disclosure relates in some aspects to methods, systems, and kits for using diphosphonic acid to promote N—O bond cleavage in a 3′ O—NH₂ nucleotide in a cell or tissue sample. In some aspects, diphosphonic acid is used to promote N—O bond cleavage in a 3′ O—NH₂ blocked reversible terminator during multiple cycles of a nucleic acid sequencing reaction in a cell or tissue sample. In some embodiments, the chemical reaction of the 3′ O—NH₂ blocked reversible terminator with diphosphonic acid releases free alcohols and phosphoric acid derivatives, which are not damaging to the cell or tissue sample.

### CROSS REFERENCE TO RELATED APPLICATIONS

This application claims priority to and the benefit of U.S. Provisional Application No. 63/697,263, filed Sep. 20, 2024, which is hereby incorporated by reference in its entirety.

### FIELD

The present disclosure relates in some aspects to methods for sequencing nucleic acid molecules, including methods for in situ sequencing and analysis of target nucleic acids in a biological sample such as a cell or tissue sample.

### BACKGROUND

Nucleic acid sequencing is a versatile tool that helps scientists advance the understanding of biology and has wide-ranging applications in various fields, such as medical diagnostics, biotechnology, forensic biology, and virology. Currently, there are several sequencing methods available, including Maxam-Gilbert sequencing, Sanger (chain-termination) sequencing, and next-generation sequencing (NGS) techniques. Despite advances in nucleic acid sequencing, many challenges remain unaddressed, particularly for performing nucleic acid sequencing in cell or tissue samples.

### SUMMARY

Nucleic acid sequencing reactions such as sequencing-by-synthesis (SBS) and sequencing-by-binding often involve use of reversible terminators for extension of priming strands in a controlled manner (e.g., only extending one base at a time). 3′ O—NH₂ is used as a common reversible terminator for nucleic acid sequencing reactions. Typically, NaNO₂ is used in the nucleic acid sequencing reaction to liberate 3′OH of the nucleotide, thereby generating a free 3′ OH that allows for further extension of the priming strand. However, the use of NaNO₂ generates tissue or cell sample damaging byproducts. There is a need for improved methods with reduced production of tissue or cell sample damaging byproducts for reactions that are performed in cell or tissue samples, such as in situ nucleic acid sequencing reactions in cell or tissue samples. The present application addresses this need by using carbonyl diphosphonic acid to promote N—O bond cleavage in a 3′ O—NH₂ nucleotide in a cell or tissue sample. In some aspects, carbonyl diphosphonic acid is used to promote N—O bond cleavage in a 3′ O—NH₂ blocked reversible terminator during multiple cycles of a nucleic acid sequencing reaction in a cell or tissue sample. In some embodiments, the chemical reaction of 3′ O—NH₂ blocked reversible terminator with carbonyl diphosphonic acid releases free alcohols and phosphoric acid derivatives, which are not damaging to the cell or tissue sample.

In some aspects, provided herein is a method of generating a nucleic acid strand comprising a free 3′-OH, comprising reacting a blocked nucleic acid strand comprising a 3′-O—NH₂ at a 3′ terminus with a diphosphonic acid of Formula (P1) or (P2):

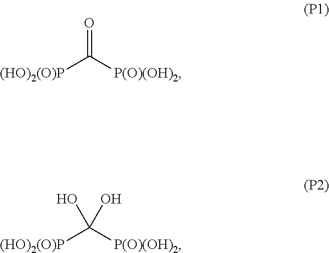

or a salt of Formula (P1) or (P2).

In some aspects, provided herein is a method of de-blocking a 3′ terminal nucleotide of a priming strand of a sequencing reaction, comprising: (a) providing a cell or tissue sample comprising: a priming strand bound to a template nucleic acid molecule, wherein the priming strand comprises a blocked nucleotide comprising a 3′-O—NH₂ at a 3′ terminus; and b) reacting the —O—NH₂ of the blocked nucleotide with a diphosphonic acid of Formula (P1) or (P2):

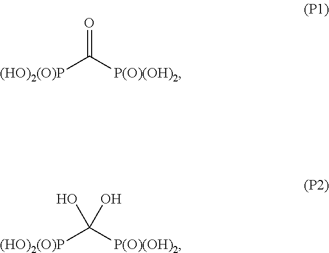

or a salt of Formula (P1) or (P2), thereby generating a free 3′-OH at the 3′ terminus of the priming strand in the cell or tissue sample.

In some aspects, provided herein is a method of sequencing at least a portion of a template nucleic acid in a cell or tissue sample, the method comprising: (a) performing a cycle of a nucleic acid sequencing reaction, comprising: using a polymerase, incorporating a blocked nucleotide comprising a 3′-O—NH₂ at a 3′ terminus into a priming strand bound to the template nucleic acid, and identifying a nucleotide in the template nucleic acid; and (b) reacting the blocked nucleotide incorporated into the priming strand with a diphosphonic acid of Formula (P1) or (P2):

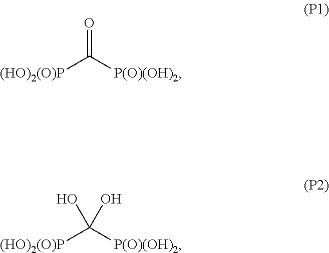

or salt of Formula (P1) or (P2), thereby generating a free 3′-OH in the priming strand in the cell or tissue sample.

In some embodiments, 3′-ONH₂ blocked nucleotide is of Formula (I):

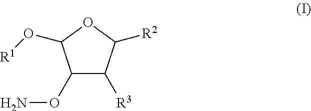

wherein R¹ is H, a monophosphate, a diphosphate, a triphosphate or a nucleic acid; R² is a nucleobase or a modified nucleobase; and R³ is H or OH. In some embodiments, R² is a modified nucleobase comprising a fluorescent group, and the method further comprises: cleaving the fluorescent group from the blocked nucleotide after (a).

In some embodiments, performing the cycle of the sequencing reaction comprises detecting the blocked nucleotide incorporated into the priming strand, thereby identifying a nucleotide at a complementary position in the template nucleic acid.

In some embodiments, performing the cycle of the sequencing reaction comprises: contacting the priming strand bound to the template with a polymerase and a free nucleotide molecule to generate a ternary polymerase complex, and detecting the ternary polymerase complex. In some embodiments, the detecting comprises detecting the free nucleotide molecule of the ternary polymerase complex, optionally wherein the free nucleotide molecule comprises or is attached to a dye moiety.

In some embodiments, the method comprises performing at least one additional cycle of the nucleic acid sequencing reaction to identify at least one additional base of the template nucleic acid, the at least one additional cycle comprises: using a polymerase, incorporating an additional blocked nucleotide comprising a 3′-O—NH₂ into the priming strand bound to the template nucleic acid to generate an extended priming strand; and identifying a nucleotide in the template nucleic acid.

In some embodiments, the at least one additional cycle comprises reacting the additional blocked nucleotide incorporated into the extended priming strand with a diphosphonic acid of Formula (P1) or (P2):

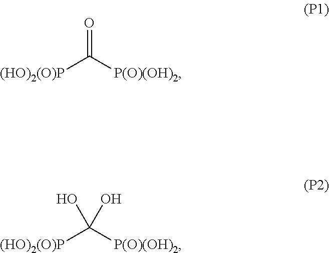

or a salt of Formula (P1) or (P2), thereby generating a free 3′-OH in the priming strand in the cell or tissue sample.

In some embodiments, the at least one additional cycle comprises at least 2, 5, 10, 20, 30, 40, or 50 additional cycles, wherein the additional cycles individually and independently comprise incorporating an additional blocked nucleotide comprising a 3′-O—NH₂ into the priming strand bound to the template nucleic acid; and identifying a nucleotide in the template nucleic acid.

In some embodiments, the additional cycles individually comprise reacting the additional 3′-O—NH₂ blocked nucleotide incorporated into the priming strand in that cycle with a diphosphonic acid of Formula (P1) or (P2):

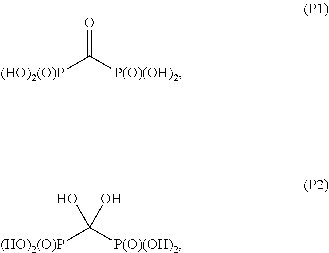

or a salt of Formula (P1) or (P2), thereby generating a free 3′-OH in the priming strand in the cell or tissue sample, before incorporating an additional blocked nucleotide comprising a 3′-O—NH₂ of a subsequent cycle.

In some embodiments, the nucleic acid sequencing reaction is performed at a pH between pH 6 and pH 10.

In some embodiments, the diphosphonic acid of Formula (P1) or (P2) or a salt of Formula (P1) or (P2) comprises a sodium salt of (P1) or (P2). In some embodiments, the sodium salt of (P1) or (P2) comprises a mono-, di-, tri-, or tetra-sodium salt of (P1) or (P2).

In some embodiments, the method comprises contacting the cell or tissue sample with a mixture of 3′-O—NH₂ blocked nucleotides comprising at least two different nucleobases selected from the group consisting of A, T or U, C, and G.

In some embodiments, the template nucleic acid comprises a DNA molecule. In some embodiments, the template nucleic acid comprises an RNA molecule. In some embodiments, the template nucleic acid comprises a target analyte nucleic acid sequence. In some embodiments, the template nucleic acid comprises a barcode sequence associated with an analyte in the cell or tissue sample.

In some embodiments, prior to the nucleic acid sequencing reaction, a circularizable probe hybridizes to a target analyte in the cell or tissue sample or to a labeling agent bound to the target analyte and is ligated to form a circularized probe, wherein the method further comprises performing rolling circle amplification of the circularized probe to generate the template nucleic acid. In some embodiments, the template nucleic acid is a rolling circle amplification product (RCP). In some embodiments, the circularizable probe is a padlock probe sequence. In some embodiments, the probe sequence comprises one or more barcode region.

In some embodiments, the target analyte comprises an mRNA molecule.

In some embodiments, the cell or tissue sample is a fresh frozen tissue section. In some embodiments, the cell or tissue sample is a paraffin embedded formalin fixed (FFPE) tissue section. In some embodiments, the cell or tissue sample is immobilized on a surface. In some embodiments, the template nucleic acid is sequenced in situ in the cell sample or tissue sample. In some embodiments, the cell or tissue sample is a cell sample comprising a layer of cells deposited on a surface.

In some embodiments, reacting the —O—NH₂ of the blocked nucleotide with the diphosphonic acid of Formula (P1) or (P2) or the salt thereof comprises contacting the cell or tissue sample with a reagent comprising the diphosphonic acid of Formula (P1) or (P2) or the salt thereof at a concentration in a range of 1 mM to 100 mM.

### BRIEF DESCRIPTION OF THE DRAWINGS

The drawings illustrate certain features and advantages of this disclosure. These embodiments are not intended to limit the scope of the appended claims in any manner.

FIG. 1 depicts a system for performing a sequencing assay, in accordance with some implementations of the methods described herein.

FIG. 2 depicts a computer system or computer network, in accordance with some instances of the systems described herein.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods for sequencing nucleic acid molecules described herein involving use of carbonyl diphosphonic acid to deblock O-substituted hydroxylamines. In some embodiments, a method provided herein comprises generating the nucleic acid strand comprising a free 3′-OH from a 3′-ONH₂ blocked nucleic acid strand in a cell or tissue sample, comprising reacting 3′-ONH₂ blocked nucleic acid strand with a diphosphonic acid of Formula (P1) or (P2), or a salt of Formula (P1) or (P2). In some embodiments, a method provided herein is a method of de-blocking a 3′ terminal nucleotide of a priming strand of a sequencing reaction, comprising: (a) providing a cell or tissue sample comprising: a priming strand bound to a template nucleic acid molecule, wherein the priming strand comprises a 3′-O—NH₂ blocked nucleotide at a 3′ terminus; and b) reacting the —O—NH₂ of the blocked nucleotide with a diphosphonic acid of Formula (P1) or (P2), or a salt of Formula (P1) or (P2), thereby generating a free 3′-OH at the 3′ terminus of the priming strand in the cell or tissue sample. In some embodiments, a method provided herein is a method of sequencing at least a portion of a template nucleic acid in a cell or tissue sample, the method comprising: (a) performing a cycle of a nucleic acid sequencing reaction, comprising: using a polymerase, incorporating a first 3′-O—NH₂ blocked nucleotide into a priming strand bound to the template nucleic acid, and identifying a nucleotide in the template nucleic acid; and (b) reacting 3′-O—NH₂ blocked nucleotide incorporated into the priming strand with a diphosphonic acid of Formula (P1) or (P2), or a salt of Formula (P1) or (P2), thereby generating a free 3′-OH in the priming strand in the cell or tissue sample.

Advantageously, the diphosphonic acid provides a way to deblock O-substituted hydroxylamines while minimizing tissue sample damage. The provided methods are particularly useful for nucleic acid sequencing reactions in cell or tissue samples. Additional aspects of the methods, compositions, kits, and systems disclosed herein are described in the sections below.

### II. Nucleic Acid Sequencing Using Diphosphonic Acid as a 3′-ONH₂ Reducing Agent

The sequencing methods described herein are useful for multi-cycle sequencing approaches where nucleotides of a template nucleic acid are “interrogated” by binding to a complementary nucleotide, or by incorporation of a complementary nucleotide into a priming strand bound to the template nucleic acid. For example, the sequencing methods described herein are applicable in situ sequencing applications (e.g., in situ sequencing of endogenous nucleic acid sequences and/or target-specific barcode sequences associated with target analytes of interest that are distributed within a cell or tissue sample). In some embodiments, the sequence of an endogenous nucleic acid or a target-specific barcode sequence is incorporated into a rolling circle amplification product in the cell or tissue sample, thereby providing multiple copies of the endogenous nucleic acid sequence or barcode sequence to be analyzed in the nucleic acid sequencing reaction. In some embodiments, the template nucleic acid is a rolling circle amplification product in the cell or tissue sample. Advantageously, the sequencing methods described herein allow for deprotection of O-substituted hydroxylamines used as reversible terminators while minimizing tissue sample damage compared to conventional methods for deprotecting O-substituted hydroxylamines.

In some embodiments, the sequencing methods described herein comprise contacting a template nucleic acid molecule with a sequencing primer designed to hybridize to a portion of the template nucleic acid molecule. In some embodiments, the sequencing primer comprises a free 3′-hydroxyl group at its 3′ terminus, and a primer extension reaction may be performed to incorporate a 3′-ONH₂ blocked nucleotide into the priming strand. In some embodiments, the method comprises comprising reacting 3′-ONH₂ blocked nucleotide in the priming strand with a diphosphonic acid of Formula (P1) or (P2), or a salt of Formula (P1) or (P2).

In some embodiments, the sequencing primer comprises a 3′-ONH₂ group. In some embodiments, the method comprises reacting 3′-ONH₂ group of the sequencing primer with a diphosphonic acid of Formula (P1) or (P2), or a salt of Formula (P1) or (P2).

In some embodiments, the In some aspects, provided herein is a method of generating the nucleic acid strand comprising a free 3′-OH from a 3′-ONH₂ blocked nucleic acid strand in a cell or tissue sample, comprising reacting 3′-ONH₂ blocked nucleic acid strand with a diphosphonic acid of Formula (P3):

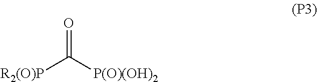

or a salt of Formula (P3).

In some aspects, provided herein is a method of de-blocking a 3′ terminal nucleotide of a priming strand of a sequencing reaction, comprising: (a) providing a cell or tissue sample comprising: a priming strand bound to a template nucleic acid molecule, wherein the priming strand comprises a 3′-O—NH₂ blocked nucleotide at a 3′ terminus; and b) reacting the —O—NH₂ of the modified nucleotide with a diphosphonic acid of Formula (P3):

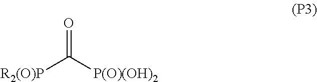

or a salt of Formula (P3), thereby generating a free 3′-OH at the 3′ terminus of the priming strand in the cell or tissue sample.

In some aspects, provided herein is a method of sequencing at least a portion of a template nucleic acid in a cell or tissue sample, the method comprising: (a) performing a cycle of a nucleic acid sequencing reaction, comprising: using a polymerase, incorporating a first 3′-O—NH₂ blocked nucleotide into a priming strand bound to the template nucleic acid, and identifying a nucleotide in the template nucleic acid; and (b) reacting 3′-O—NH₂ blocked nucleotide incorporated into the priming strand with a diphosphonic acid of Formula (P3):

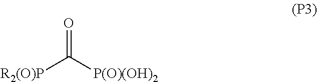

or a salt of Formula (P3), thereby generating a free 3′-OH in the priming strand in the cell or tissue sample.

In some embodiments, the diphosphonic acid of Formula (P3) or salt of Formula (P3) comprises a sodium salt of (P3). In some embodiments, the sodium salt of (P3) comprises a mono-, di-, tri-, or tetra-sodium salt of (P3).

In some embodiments, reacting the diphosphonic acid or salt thereof with 3′-ONH₂ comprises incubating the diphosphonic acid or salt thereof with the cell or tissue sample at around 20° C. (e.g., at a temperature between about 10° C. and about 37° C., between about 15° C. and about 30° C., between about 15° C. and about 25° C., or between about 18° C. and about 22° C.). In some embodiments, reacting the diphosphonic acid or salt thereof with 3′-ONH₂ comprises incubating the diphosphonic acid or salt thereof with the cell or tissue sample for at least 1 minute. In some embodiments, reacting the diphosphonic acid or salt thereof with 3′-ONH₂ comprises incubating the diphosphonic acid or salt thereof with the cell or tissue sample for between about 1 minute and about 5 minutes, or for between about 1 minute and about 2 minutes. In some embodiments, reacting the diphosphonic acid or salt thereof with 3′-ONH₂ comprises incubating the diphosphonic acid or salt thereof with the cell or tissue sample in an aqueous buffer having a pH between about 6 and about 10. In some embodiments, the aqueous buffer has a pH between about pH 6 and pH 9, between about pH 7 and pH 9, or between about pH 7 and pH 8. In some embodiments, the diphosphonic acid is carbonyl diphosphonic acid.

In some embodiments, reacting 3′-O—NH₂ blocked nucleotide incorporated into the priming strand with the diphosphonic acid comprises contacting the cell or tissue sample with a composition comprising the diphosphonic acid at a concentration in a range of 100 μM to 100 mM. In some embodiments, the diphosphonic is at a concentration in a range of 100 μM to 50 mM, 100 μM to 40 mM, 100 μM to 30 mM, 100 μM to 20 mM, or 100 μM to 10 mM. In some embodiments, the diphosphonic is at a concentration in a range of 500 μM to 100 mM, 600 μM to 100 mM, 700 μM to 100 mM, 800 μM to 100 mM, 900 μM to 100 mM, or 1 mM to 100 mM. In some embodiments, the diphosphonic is at a concentration in a range of 1 mM to 50 mM. In some embodiments, the diphosphonic is at a concentration in a range of 10 mM to 50 mM.

In some embodiments, the diphosphonic acid reacts with 3′-ONH₂ blocked nucleotide to form an intermediate compound of Formula (O):

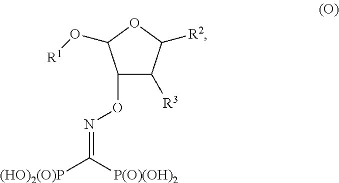

or a salt thereof, wherein R¹ is a nucleic acid; R² comprises a nucleobase or a modified nucleobase; and R³ is H or OH. In some aspects, provided herein is a cell or tissue sample comprising a compound of Formula (O) or a salt thereof.

In some embodiments, the sequencing method described herein comprises sequencing-by-synthesis. In some embodiments, the sequencing method described herein comprises performing a cyclic series of base-by-base sequencing reactions, where each sequencing cycle comprises:
(a) contacting the priming strand bound to the template nucleic acid molecule with a polymerase and a composition comprising 3′-ONH₂ blocked nucleotides, wherein each 3′-ONH₂ blocked nucleotide comprises a different nucleobase selected from A, T or U, G, and C;
(b) incorporating one of 3′-ONH₂ blocked nucleotides of the composition of 3′-ONH₂ blocked nucleotides into the priming strand to form an extended priming strand;
(c) detecting the incorporated 3′-ONH₂ blocked nucleotide to identify a complementary nucleotide in the template nucleic acid molecule; and
(d) reacting the incorporated 3′-ONH₂ blocked nucleotide with a diphosphonic acid of Formula (P1) or (P2), or a salt of Formula (P1) or (P2). In some embodiments, the diphosphonic acid of Formula (P1) or (P2) or a salt of Formula (P1) or (P2) comprises a sodium salt of (P1) or (P2). In some embodiments, the sodium salt of (P1) or (P2) comprises a mono-, di-, tri-, or tetra-sodium salt of (P1) or (P2). In some embodiments, the method comprises performing at least 2, 3, 4, 5, 6, 7, 8, 9, 10, 15, 20, 25, 30, 35, 40, or more additional cycles of the nucleic acid sequencing reaction.

In some embodiments, detecting the presence of the incorporated 3′-ONH₂ blocked nucleotide comprises detecting a fluorescence signal (or combination of fluorescent signals) associated with a particular nucleobase. In some embodiments, identifying a complementary nucleotide in the template nucleic acid molecule comprises detecting an absence of a signal (e.g., an unlabeled 3′-ONH₂ blocked nucleotide of the composition is incorporated into the priming strand to form an extended priming strand). In some embodiments, at least three of the different 3′-ONH₂ blocked nucleotides are labeled with different fluorescent dyes, thereby associating different fluorescent signals with three different nucleobases (and absence of signal with a fourth nucleobase). In some embodiments, the composition comprising 3′-ONH₂ blocked nucleotides comprises a first 3′-ONH₂ blocked nucleotide labeled with a first fluorescent dye, a second 3′-ONH₂ blocked nucleotide labeled with a second fluorescent dye, a mixture of a third 3′-ONH₂ blocked nucleotide labeled with the first fluorescent dye and the third 3′-ONH₂ blocked nucleotide labeled with the second fluorescent dye, and a fourth 3′-ONH₂ blocked nucleotide that is unlabeled (e.g., for two-color nucleic acid sequencing). In some embodiments, the composition comprising 3′-ONH₂ blocked nucleotides comprises four different 3′-ONH₂ blocked nucleotides, each labeled with a different fluorescent dye. In some embodiments, after detecting the incorporated 3′-ONH₂ blocked nucleotide, the method comprises cleaving the fluorescent dye from the incorporated nucleotide.

In some embodiments, the sequencing method described herein comprises sequencing-by-binding or sequencing-by-avidity. In some embodiments, the sequencing method described herein comprises performing a cyclic series of base-by-base sequencing reactions, where each sequencing cycle comprises:
(a) contacting the priming strand bound to the template nucleic acid molecule with a polymerase and a composition comprising nucleotides or nucleotide complexes for sequencing for avidity, wherein each nucleotide or nucleotide complex comprises a different nucleobase selected from A, T or U, G, and C, wherein the priming strand comprises a 3′-ONH₂;
(b) detecting a ternary complex formed between one of the nucleotides or nucleotide complexes, the polymerase, and the template nucleic acid to identify a complementary nucleotide in the template nucleic acid molecule; and
(c) reacting 3′-ONH₂ in the priming strand with a diphosphonic acid of Formula (P1) or (P2), or a salt of Formula (P1) or (P2). In some embodiments, the diphosphonic acid of Formula (P1) or (P2) or salt of Formula (P1) or (P2) comprises a sodium salt of (P1) or (P2). In some embodiments, the sodium salt of (P1) or (P2) comprises a mono-, di-, tri-, or tetra-sodium salt of (P1) or (P2). In some embodiments, an individual cycle of the sequencing reaction further comprises (d) incorporating a 3′-ONH₂ blocked nucleotide into the priming strand to generate an extended priming strand comprising a 3′-ONH₂. In some embodiments, the method comprises performing at least 2, 3, 4, 5, 6, 7, 8, 9, 10, 15, 20, 25, 30, 35, 40, or more additional cycles of the nucleic acid sequencing reaction.

In some embodiments, detecting the ternary complex comprises detecting a fluorescence signal (or combination of fluorescent signals) associated with a particular nucleobase of the composition. In some embodiments, identifying a complementary nucleotide in the template nucleic acid molecule comprises detecting an absence of a signal (e.g., an unlabeled nucleotide of the composition forms a ternary complex with the template nucleic acid). In some embodiments, at least three of the different nucleotides or complexes are labeled with different fluorescent dyes, thereby associating different fluorescent signals with three different nucleobases (and absence of signal with a fourth nucleobase). In some embodiments, the composition comprising different nucleotides or complexes comprises a first nucleotide or complex labeled with a first fluorescent dye, a second nucleotide or complex labeled with a second fluorescent dye, a mixture of a third nucleotide or complex labeled with the first fluorescent dye and the third nucleotide or complex labeled with the second fluorescent dye, and a fourth nucleotide or complex that is unlabeled (e.g., for two-color nucleic acid sequencing). In some embodiments, the composition comprising different nucleotides comprises four different nucleotides or complexes, each labeled with a different fluorescent dye.

In some embodiments, 3′-ONH₂ nucleotide is provided in an oxime-protected form. In some embodiments, the oxime-protected form of 3′-ONH₂ nucleotide comprises a 3′-O—N═CR₂, wherein R is C₁-C₆ alkyl. In some embodiments, the oxime-protected form is converted to 3′-ONH₂ before contacting the cell or tissue sample. In some embodiments, the method comprises contacting the cell or tissue sample with the oxime-protected form of 3′-ONH₂ blocked nucleotide, and converting it into 3′-ONH₂ blocked nucleotide.

### Template Nucleic Acid Molecules

In some embodiments, the template nucleic acid molecule includes a target analyte nucleic acid molecule (e.g., a DNA molecule, an RNA molecule, or an mRNA molecule). In some embodiments, the template nucleic acid includes a reporter oligonucleotide, such as a barcode.

In some embodiments, the template nucleic acid molecule is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. In some embodiments, the DNA molecule is copied from another nucleic acid molecule (e.g., DNA or RNA such as mRNA).

In some embodiments, the template nucleic acid molecule is an RNA molecule. Examples of RNA template nucleic acid molecules include RNA molecules such as various types of coding and non-coding RNA. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at 3′ end), and a spliced mRNA in which one or more introns have been removed. Also included in the analytes disclosed herein are non-capped mRNA, a non-polyadenylated mRNA, and a non-spliced mRNA. In some embodiments, the RNA template nucleic acid molecule is copied from another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

In some embodiments, the template nucleic acid comprises a nucleic acid analyte derived from a biological sample and/or a reporter oligonucleotide or nucleic acid marker associated with an analyte from a biological sample. Such analytes can be or derived from any biological sample. In some embodiments, the template nucleic acid comprises a nucleic acid analyte and/or a reporter oligonucleotide or nucleic acid marker associated with an analyte present in a biological sample, and the template nucleic acid molecule is sequenced at a location in the biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, in some embodiments, a biological sample is obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample from an organism comprises one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained are healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, a template nucleic acid includes a reporter oligonucleotide or marker associated with the presence of an analyte (e.g., an endogenous analyte) in a sample. Such analytes may include nucleic acid analytes and/or non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some embodiments, a template nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, methods and compositions disclosed herein are used to analyze any number of template nucleic acid molecules (e.g., nucleic acid analytes and/or analyte-associated barcode sequences) or fragments thereof. For example, in some embodiments, the number of analytes that are analyzed is at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of a sample (e.g., a cell sample or tissue sample) or tethered within individual features on a substrate (e.g., a flow cell surface).

### Nucleotide Compositions for Sequencing

In some embodiments, each cycle of a cyclic series of base-by-base sequencing reactions performed as part of the disclosed methods comprises contacting priming strands bound to template nucleic acid molecules with a composition comprising nucleotides. In some embodiments, each cycle of a cyclic series of base-by-base sequencing reactions comprises contacting the priming strand bound to a template nucleic acid molecule with a composition comprising a plurality of sets of nucleotides (e.g., 2, 3, or 4 sets of nucleotides). In some embodiments, each set nucleotides comprises a same nucleobase that differs from the other sets.

In some embodiments, nucleotides of a composition contacted with the primed template nucleic acid molecule(s) in each cycle of a multicycle sequencing process comprise A, T, U, C, and/or G. In some embodiments, nucleotides of a composition contacted with the primed template nucleic acid molecule(s) in each cycle of a multicycle sequencing process comprise A, T, C, and/or G.

In some embodiments, the composition comprising nucleotides contacted with the primed template nucleic acid molecule(s) is the same in each cycle of a multicycle sequencing process (e.g., the composition comprises the same sets of nucleotides in each cycle, wherein each set of nucleotides comprises the same selection of A, T, U, C, and/or G).

In some embodiments, the composition comprising nucleotides contacted with the primed template nucleic acid molecule(s) is different between at least 2 cycles of a multicycle sequencing process (e.g., the composition comprises different sets of nucleotides in different cycles, wherein the sets of nucleotides comprise a different selection of A, T, U, C, and/or G, and/or wherein the sets of nucleotides comprise a different selection fluorophores).

In some embodiments, a composition comprises 4 sets of nucleotides, wherein each set comprises a same nucleobase (e.g., selected from A, T, U, C, and/or G) that differs from the other sets, and wherein, for each set, nucleotides are labeled with a same fluorophore that differs from the other sets.

In some embodiments, a composition comprises a set of unlabeled nucleotides (e.g., not conjugated to a fluorophore) in addition to labeled nucleotides or complexes. In some embodiments, a set of unlabeled nucleotides is used to implement different detection schemes (e.g., two color, three color, or four color detection schemes). In some embodiments, a composition comprises three sets of nucleotides, each set comprising a same nucleobase (e.g., selected from A, T, U, C, and/or G) that differs from the other sets, and wherein, for each set, each set of nucleotides comprises a same fluorophore that differs from the other sets, and wherein the composition further comprises a set of unlabeled nucleotides comprising a same nucleobase that differs from the nucleobase of the three sets of nucleotides. In some embodiments, a set of unlabeled nucleotides is used to implement the readout of target-specific barcode designs used to minimize optical crowding when performing in situ sequencing (see, e.g., PCT International Patent Application Publication Nos. WO 2022/060889 and WO 2023/220300, each of which is incorporated herein by reference in its entirety).

In some embodiments, the labeled and/or unlabeled nucleotides in the composition are the 3′-ONH₂ blocked nucleotides. In some embodiments, the composition of nucleotides is a composition for sequencing-by-binding or sequencing-by-avidity, and the method further comprises contacting the priming strands bound to template nucleic acids with a separate composition comprising 3′-ONH₂ nucleotides. In some embodiments, 3′-ONH₂ nucleotides of a composition contacted with the primed template nucleic acid molecule(s) in each cycle of a multicycle sequencing process comprise A, T, U, C, and/or G. In some embodiments, 3′-ONH₂ nucleotides of a composition contacted with the primed template nucleic acid molecule(s) in each cycle of a multicycle sequencing process comprise A, T, C, and/or G.

In some embodiments, 3′-ONH₂ blocked nucleotide is of Formula (I):

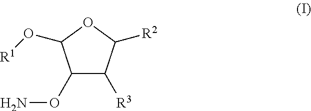

wherein R¹ is H, a monophosphate, a diphosphate, a triphosphate or a nucleic acid; R² is a nucleobase or a modified nucleobase; and R³ is H or OH. In some embodiments, R² is a modified nucleobase comprising a fluorescent group, and the method further comprises: cleaving the fluorescent group from 3′-O-blocked sequence after (a). In some embodiments, the cleaving is before (b). In some embodiments, the cleaving is after (b).

### Primers (or Primer Sequences)

In some instances, the disclosed methods for performing nucleic acid sequencing (e.g., in situ sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Polymerases

Examples of polymerases that are used for performing the disclosed methods include, but are not limited to, DNA polymerases (e.g., Taq DNA polymerase), RNA polymerases, and/or reverse transcriptases.

In some embodiments, the polymerase is a DNA polymerase. Examples of DNA polymerases include Taq polymerase, 9°N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some embodiments, DNA polymerases that have been engineered or mutated to have desirable characteristics are employed. In some embodiments, the polymerase is phi29 DNA polymerase. In some embodiments, the polymerase is a DNA polymerase, and the template nucleic acid molecule includes DNA. In some embodiments, the polymerase is a DNA polymerase, and the nucleotide molecules include deoxyribonucleotides.

In some embodiments, the DNA polymerase is Taq polymerase or a functional variant thereof. Taq polymerase is a heat stable polymerase from Thermus aquaticus.

In some embodiments, the DNA polymerase is a 9°N-7 DNA polymerase or a functional variant thereof (e.g., D141A/E143A/A485L). 9°N-7 is a strain of Thermococcus sp.

In some embodiments, the DNA polymerase is DNA polymerase I or a functional fragment thereof (e.g., a Klenow fragment). Klenow fragment is an exonuclease deficient fragment of DNA polymerase I.

In some embodiments, the polymerase is a reverse transcriptase. Reverse transcriptases typically have RNA-dependent DNA polymerase activity and DNA-dependent DNA polymerase activity. Examples of reverse transcriptases include Moloney murine leukemia virus (MMLV) reverse transcriptase, HIV-1 reverse transcriptase, and avian myeloblastosis virus (AMV) reverse transcriptase. In some embodiments, the reverse transcriptase lacks (e.g., is mutated to lack) ribonuclease activity. In some embodiments, ribonuclease activity degrades template particularly during longer incubation times such as when reverse transcribing longer cDNAs. In some embodiments, the polymerase is a reverse transcriptase, and the template nucleic acid molecule is an RNA molecule. In some embodiments, the polymerase is a reverse transcriptase, and the nucleotide molecules include deoxyribonucleotide molecules.

In some embodiments, the reverse transcriptase is an MMLV reverse transcriptase or a functional variant thereof.

In some embodiments, the reverse transcriptase is an HIV-1 reverse transcriptase or a functional variant thereof.

In some embodiments, the polymerase is selected from Taq polymerase, 9°N-7 DNA polymerase or a functional variant thereof (e.g., D141A/E143A/A485L), and a Klenow fragment of DNA polymerase I.

### Base-Calling

As noted elsewhere herein, the disclosed methods for performing nucleic acid sequencing (e.g., in situ sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5): 786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory-European Bioinformatics Institute, Cambridgeshire, UK), and Bustard (Illumina, Inc., San Diego, CA).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for nucleic acid sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for nucleic acid sequencing comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for nucleic acid sequencing comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for nucleic acid sequencing comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5): 455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14): 1838-1844), and String Graph Assembler (SGA) (see, e.g., Simpson et al. (2012), “Efficient de novo Assembly of Large Genomes Using Compressed Data Structures”, Genome Res. 22:549-556).

### Workflows for Performing Sequencing

The disclosed sequencing methods may be applied to both in situ sequencing and flow cell sequencing applications, where the sequencing reactions described involving reaction of the diphosphonic acid with 3′-ONH₂ are used rather than a conventional sequencing-by-synthesis (SBS), sequencing-by-binding, or sequencing-by-avidity method.

In the case of in situ sequencing, in some embodiments, the disclosed methods comprise performing all or a subset of the following steps (in addition to a cyclic series of base-by-base sequencing reactions involving reaction of diphosphonic acid with a 3′-ONH₂):
(i) preparing the biological sample (e.g., by fixing, sectioning, embedding, and/or clearing a cell or tissue sample, as described elsewhere herein).
(ii) contacting target analytes (e.g., target nucleic acid analytes and/or protein analytes) within the prepared sample with target-specific probes, as described elsewhere herein. In some embodiments, the target-specific probes comprise, e.g., target-specific linear and/or circularizable nucleic acid probes (e.g., padlock probes) designed to hybridize directly or indirectly to specific target nucleic acid analytes. In some embodiments, the target-specific linear and/or circularizable nucleic acid probes comprise primer binding sites and/or target-specific barcode (or identifier) sequences. In some embodiments, the target-specific probes comprise, e.g., target-specific antibodies designed to bind to specific target protein analytes, where the antibodies are conjugated to nucleic acid sequences. In some embodiments, the conjugated nucleic acid sequences comprise primer binding sites and/or target-specific barcode (or identifier) sequences.
(iii) optionally performing a reverse transcription reaction (e.g., if the probed target nucleic acid analytes comprise RNA molecules) to create cDNA copies of RNA target molecules.
(iv) optionally amplifying the probed target analyte molecules and/or their associated target-specific barcode sequences (e.g., using rolling circle amplification (RCA) in the case that target-specific circularizable probes were used to probe target analyte molecules and/or associated barcode sequences).
(v) contacting the optionally amplified target nucleic acid analytes and/or associated target-specific barcode sequences with sequencing primers designed to hybridize directly or indirectly to the target nucleic acid analytes and/or their associated target-specific barcode sequences. In some embodiments, the sequencing primers comprise free 3′-hydroxyl groups at their 3′ termini, and a primer extension reaction is performed to incorporate a single 3′-ONH₂ blocked nucleotide at 3′ termini of a bound primer (i.e., 3′ termini of the priming strands). In some embodiments, the sequencing primers comprise 3′-ONH₂ groups at their 3′ termini.

In some embodiments, the disclosed methods comprise: performing a cyclic series of base-by-base sequencing reactions, where each sequencing cycle comprises:
(a) contacting each priming strand bound to a template nucleic acid molecule with a polymerase and a first composition comprising 3′-ONH₂ blocked nucleotides;
(b) incorporating one of 3′-ONH₂ blocked nucleotides into the priming strand to form an extended priming strand; and
(c) detecting the incorporated 3′-O NH₂ blocked nucleotide in the extended priming strand, thereby identifying a nucleotide at a complementary position in the template nucleic acid; or
performing a sequencing-by-binding or sequencing-by-avidity reaction to identify a nucleotide in the template nucleic acid 5′ and adjacent to the template nucleic acid sequence bound to the extended priming strand.

In some embodiments, the disclosed methods further comprise processing optical signals (e.g., fluorescence signals) detected in images (e.g., fluorescence images) acquired during the cyclic series of base-by-base sequencing reactions to detect the presence or absence of complementary nucleotides in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences).

In some embodiments, the cyclic series of base-by-base sequencing reactions comprises performing at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the base-by-base sequencing reaction.

In some embodiments, each cycle of base-by-base sequencing further comprises a first wash step following the contacting step to remove unbound polymerase and nucleotides. In some embodiments, the first wash step comprises, for example, use of the same buffer used for contacting the primed template nucleic acid with a polymerase and a composition comprising nucleotides (but without the polymerase and composition). In some embodiments, the first wash buffer may not include KCl and/or may include little to no DMSO. In some embodiments, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, each cycle of base-by-base sequencing further comprises removing the fluorophore from incorporated nucleotides following the detection step. In some embodiments, the fluorophore is attached to the incorporated nucleotide via a cleavable linker. In some embodiments, each cycle of base-by-base sequencing further comprises cleaving the linker between a fluorophore and the incorporated nucleotide, if the incorporated nucleotide comprises a fluorophore. Exemplary cleavable linkers and reaction conditions for cleaving such linkers are described elsewhere herein.

In some embodiments, each cycle of base-by-base sequencing further comprises photobleaching the fluorophore the incorporated nucleotide following the detection step. In some embodiments, the fluorophore of the nucleotide is unable to fluoresce after photobleaching. Any suitable photobleaching methods can be implemented. In some embodiments, the sample is exposed to a light source until the signal emitted by the fluorophore is eliminated.

In some embodiments, the detection step comprises the use of an optical imaging technique (e.g., a fluorescence imaging technique) and real time or post-processing measurement of optical signals (e.g., fluorescence signals or the absence thereof) associated with the presence of a specific nucleotide at a plurality of locations corresponding to a plurality of target analytes distributed throughout the biological sample or tethered to specific locations on a substrate surface (e.g., a flow cell surface).

In some embodiments, the template nucleic acid molecule comprises an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some embodiments, the template nucleic acid molecule comprises a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some embodiments, the method further comprises hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some embodiments, for example, the circularizable probe is a padlock probe sequence.

In some embodiments, the template nucleic acid molecule is sequenced in situ in a cell sample or tissue sample. In some embodiments, the cell sample comprises a layer of cells deposited on a surface.

### III. Biological Samples & Sample Preparation

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample is prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein comprise one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein comprises de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and Rnase inactivating agents or inhibitors such as proteinase K, and/or chelating agents such as EDTA, can be added to the sample. For example, a method disclosed herein comprises a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix comprises a network of natural molecules and/or synthetic molecules that are chemically and/or enzymatically linked, e.g., by crosslinking. In some instances, a 3D matrix comprises a synthetic polymer. In some instances, a 3D matrix comprises a hydrogel.

In some embodiments, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample.

In some instances, the biological sample is immobilized in the hydrogel via cross-linking of the polymer material that forms the hydrogel. Cross-linking can be performed chemically and/or photochemically, or alternatively by any other suitable hydrogel-formation method.

In some instances, the biological sample is reversibly cross-linked prior to or during an in situ assay. In some embodiments, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof can be modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly (hydroxyethyl acrylate), and poly (hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221): 543-548, 2015, the entire contents of which are incorporated herein by reference.

In some instances, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) can be isometrically expanded. Isometric expansion methods that can be used include hydration, a preparative step in expansion microscopy, as described in, e.g., Chen et al., Science 347(6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

### Staining and Immunohistochemistry (IHC)

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, SSDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### IV. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some embodiments, an analyte can include any biological substance, structure, moiety, or component to be analyzed. In some embodiments, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of an RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer.

Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

### Labeling Agents

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some embodiments, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Detectable Labels

In some instances, one or more hybridization probes or one or more nucleotides (or analogs thereof) can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some instances, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

In some instances, a detectable label can be attached to another moiety, for example, a nucleotide or nucleotide analog. In some instances, one or more nucleotides can be labeled with a cleavable detectable tag or label. For example, the non-terminating fluorescently labeled nucleotides can include a DBCO-nucleotide conjugated to fluorescent compound with a disulfide linker. In some instances, a non-terminating fluorescently labeled nucleotide is incorporated into the strand without termination, and after imaging, the linker can be cleaved to remove fluorescent label. In some instances, a DBCO-nucleotide (e.g., 5-DBCO-PEG₄-UTP) can undergo a click reaction with the cleavable linker conjugated to a fluorescent label (e.g., cleavable linker-ATTO647N), and a disulfide group can be cleaved by tris (2-carboxyethyl) phosphine (TCEP) reduction together with 3′-O-azidomethyl-dNTP.

In some instances, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, CI-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Bluc, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Bluc®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Bluc, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARFR-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically cleavable (e.g., via a reducing agent, such as dithiothreitol (DTT), tris (2-carboxyethyl) phosphine (TCEP)) or enzymatically cleavable (e.g., via an esterase, lipase, peptidase, or protease).

### Generation of Products

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some instances, a ligation product of an endogenous analyte and/or a labeling agent can be analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, provided herein is a probe or probe set capable of DNA-templated ligation, such as from a cDNA molecule. See, e.g., U.S. Pat. No. 8,551,710, which is hereby incorporated by reference in its entirety. In some instances, provided herein is a probe or probe set capable of RNA-templated ligation. See, e.g., U.S. Pat. Pub. 2020/0224244 which is hereby incorporated by reference in its entirety. In some instances, the probe set is a SNAIL probe set. Sec, e.g., U.S. Pat. Pub. 20190055594, which is hereby incorporated by reference in its entirety. In some instances, provided herein is a multiplexed proximity ligation assay. Sec, e.g., U.S. Pat. Pub. 20140194311 which is hereby incorporated by reference in its entirety. In some instances, provided herein is a probe or probe set capable of proximity ligation, for instance a proximity ligation assay for RNA (e.g., PLAYR) probe set. Sec, e.g., U.S. Pat. Pub. 20160108458, which is hereby incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. Sec, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76(14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves enzymatic ligation. In some instances, the enzymatic ligation involves use of a ligase. In some embodiments, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9°N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has a DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo) nucleotides) or by the extension of 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending 3′ end of a polynucleotide. In some cases, ligation involves ligating the ends of the probe to at least one gap (oligo) nucleotide, such that the gap (oligo) nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some embodiments, ligation stabilizes the hybridization complex containing the ligated polynucleotides prior to subsequent steps, comprising amplification and detection.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some instances, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some embodiments, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (Sec, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). RCA may be performed using a strand-displacing polymerase. Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some instances, the polymerase is phi29 DNA polymerase.

In some embodiments, during the amplification step, modified nucleotides can be added to the reaction to incorporate the modified nucleotides in the amplification product (e.g., nanoball). Non-limiting examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some embodiments, the amplification products comprise a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### VI. Fluorescence Detection and Imaging

### Fluorescence Detection

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some instances, non-limiting examples of techniques and methods applicable to the provided embodiments comprise those described in, for example, U.S. Pat. Nos. 4,757,141, 5,151,507 and 5,091,519. In some instances, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### Imaging

In some embodiments, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some embodiments, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a simpler set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) are quantified and analyzed.

### V. Barcode Sequences

In some instances, an analyte described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some embodiments, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding instances, the methods provided herein can include analyzing the barcodes by performing sequential hybridization and detection with a plurality of labelled probes (e.g., detection oligos) or by performing in situ sequencing.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4N unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### VI. Opto-Fluidic Instrument

In some instances, the sequencing methods described herein (e.g., in situ sequence or flow cell sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., compositions comprising nucleotides, primers, detectable-labeled probes and/or non-labeled probes, polymerases and/or other enzymes, deprotection reagents, buffers, etc.) to the biological sample (e.g., to a sample cartridge within which the biological sample is contained) or to a flow cell (e.g., within which nucleic acid molecules extracted from the biological sample have been tethered) and/or to remove spent reagents therefrom. In some instances, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more composition (e.g., composition nucleotides, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample. In some instances, the fluidics module is configured to deliver one or more compositions (e.g., compositions of nucleotides, as well as primers, polymerases, deprotection reagents, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface are processed in real time and/or at a later time to determine the sequence of the one or more nucleic acid sequences (e.g., barcode sequences associated with one or more target molecules) that have been extracted from a biological sample. In some embodiments, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

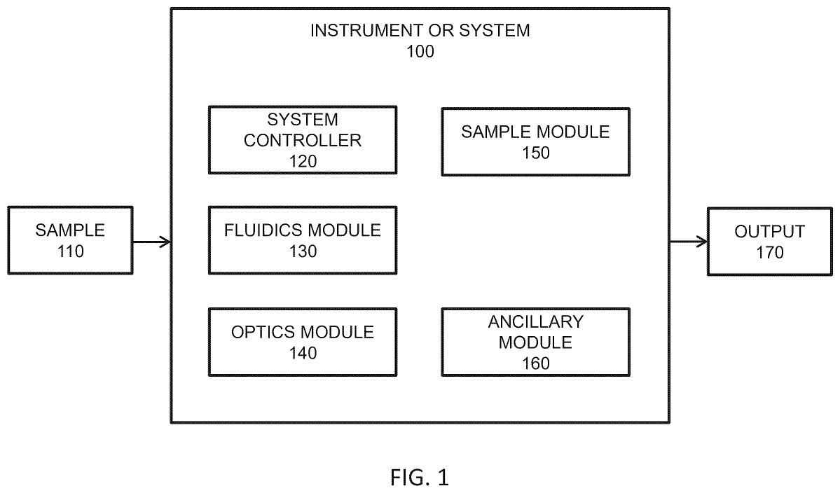

FIG. 1 shows an example workflow of analysis of a biological sample 110 (e.g., cell or tissue sample) using an opto-fluidic instrument or system 100, according to various instances. In various instances, the sample 110 can be a biological sample (e.g., a tissue) that includes molecules such as DNA, RNA, proteins, antibodies, etc. For example, the sample 110 can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various instances, the sample 110 may be placed in the opto-fluidic instrument or system 100 for analysis and detection of the molecules in the sample 110. In various instances, the opto-fluidic instrument or system 100 can be a system configured to facilitate the experimental conditions conducive for the detection of the target molecules. For example, the opto-fluidic instrument or system 100 can include a fluidics module 130, an optics module 140, a sample module 150, and an ancillary module 160, and these modules may be operated by a system controller 120 to create the experimental conditions for hybridization probe-based detection and/or base-by-base sequencing of nucleic acid molecules in the sample 110, as well as to facilitate the imaging of the sample (e.g., by an imaging system of the optics module 140). In various instances, the various modules of the opto-fluidic instrument or system 100 may be separate components in communication with each other, or at least some of them may be integrated together.

In various instances, the sample module 150 may be configured to receive the sample 110 into the opto-fluidic instrument or system 100. For instance, the sample module 160 may include a sample interface module (SIM) that is configured to receive a sample device (e.g., cassette) onto which the sample 110 can be deposited. That is, the sample 110 may be placed in the opto-fluidic instrument or system 100 by depositing the sample 110 (e.g., the sectioned tissue) on a sample device that is then inserted into the SIM of the sample module 150. In some instances, the sample module 150 may also include an X-Y stage onto which the SIM is mounted. The X-Y stage may be configured to move the SIM mounted thereon (e.g., and as such the sample device containing the sample 110 inserted therein) in perpendicular directions along the two-dimensional (2D) plane of the opto-fluidic instrument or system 100.

The experimental conditions that are conducive for the detection of the molecules in the sample 110 may depend on the target molecule detection technique that is employed by the opto-fluidic instrument or system 100. For example, in various instances, the opto-fluidic instrument or system 100 can be a system that is configured to detect molecules (e.g., by detecting hybridization probes that hybridize to nucleic molecules (e.g., barcode sequences) and/or by nucleotides incorporated into extending sequencing primers using an identifier sequence as a template) in the sample 110.

In various instances, the fluidics module 130 may include one or more components that may be used for storing the reagents, as well as for transporting said reagents to and from the sample device containing the sample 110. For example, the fluidics module 130 may include reservoirs configured to store the reagents, as well as a waste container configured for collecting the reagents (e.g., and other waste) after use by the opto-fluidic instrument or system 100 to analyze and detect the molecules of the sample 110. Further, the fluidics module 130 may also include pumps, tubes, pipettes, etc., that are configured to facilitate the transport of the reagent to the sample device (e.g., and as such the sample 110). For instance, the fluidics module 130 may include pumps (“reagent pumps”) that are configured to pump washing/stripping reagents to the sample device for use in washing/stripping the sample 110 (e.g., as well as other washing functions such as washing an objective lens of the imaging system of the optics module 140).

In various instances, the ancillary module 160 can be a cooling system of the opto-fluidic instrument or system 100, and the cooling system may include a network of coolant-carrying tubes that are configured to transport coolants to various modules of the opto-fluidic instrument or system 100 for regulating the temperatures thereof. In such cases, the fluidics module 130 may include coolant reservoirs for storing the coolants and pumps (e.g., “coolant pumps”) for generating a pressure differential, thereby forcing the coolants to flow from the reservoirs to the various modules of the opto-fluidic instrument or system 100 via the coolant-carrying tubes. In some instances, the fluidics module 130 may include returning coolant reservoirs that may be configured to receive and store returning coolants, e.g., heated coolants flowing back into the returning coolant reservoirs after absorbing heat discharged by the various modules of the opto-fluidic instrument or system 100. In such cases, the fluidics module 130 may also include cooling fans that are configured to force air (e.g., cool and/or ambient air) into the returning coolant reservoirs to cool the heated coolants stored therein. In some instances, the fluidics module 130 may also include cooling fans that are configured to force air directly into a component of the opto-fluidic instrument or system 100 so as to cool said component. For example, the fluidics module 130 may include cooling fans that are configured to direct cool or ambient air into the system controller 120 to cool the same.

As discussed above, the opto-fluidic instrument or system 100 may include an optics module 140 which include the various optical components of the opto-fluidic instrument or system 100, such as but not limited to a camera, an illumination module (e.g., LEDs), an objective lens, and/or the like. The optics module 140 may include a fluorescence imaging system that is configured to image the fluorescence emitted by the detectably labeled nucleotides are incorporated in extending sequencing primers in the sample 110 after the detectable labels are excited by light from the illumination module of the optics module 140.

In some instances, the optics module 240 may also include an optical frame onto which the camera, the illumination module, and/or the X-Y stage of the sample module 150 may be mounted.

In various instances, the system controller 120 may be configured to control the operations of the opto-fluidic instrument or system 100 (e.g., and the operations of one or more modules thereof). In some instances, the system controller 120 may take various forms, including a processor, a single computer (or computer system), or multiple computers in communication with each other. In various instances, the system controller 120 may be communicatively coupled with data storage, set of input devices, display system, or a combination thereof. In some cases, some or all of these components may be considered to be part of or otherwise integrated with the system controller 120, may be separate components in communication with each other, or may be integrated together. In other examples, the system controller 120 can be, or may be in communication with, a cloud computing platform.

In various instances, the opto-fluidic instrument or system 100 may analyze the sample 110 and may generate the output 170 that includes indications of the presence of the target molecules in the sample 110. For instance, with respect to instances discussed above where the opto-fluidic instrument or system 100 employs a sequencing technique for detecting molecules, the opto-fluidic instrument or system 100 may cause the sample 110 to undergo successive sequencing cycles, where during the same sequencing cycle the sample is imaged to detect signals associated with nucleotide binding and/or incorporation events at some locations in the sample 110, as well as to detect an absence of signals at other locations in the sample. In such cases, the output 170 may include a series of optical signals (e.g., a code word) specific to each identifier sequence (e.g., a barcode sequence), which allow the identification of the target molecules.

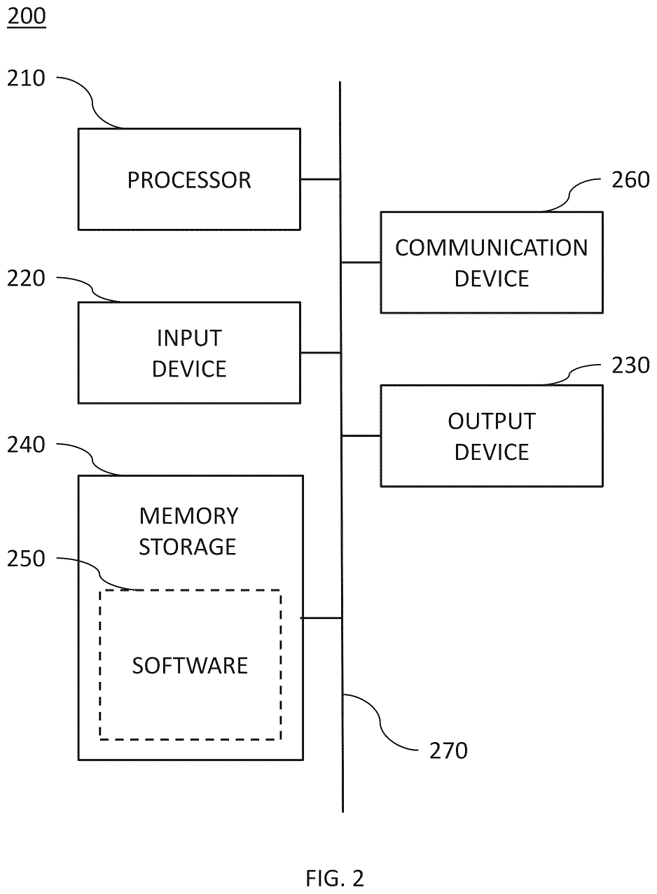

FIG. 2 illustrates an example of a computing device or system in accordance with one or more examples of the disclosure. Device 200 can be a host computer connected to a network. Device 200 can be a client computer or a server. As shown in FIG. 2, device 200 can be any suitable type of microprocessor-based device, such as a personal computer, workstation, server, or handheld computing device (portable electronic device), such as a phone or tablet. The device can include, for example, one or more of processor 210, input device 220, output device 230, memory/storage 240, and communication device 260. Input device 220 and output device 230 can generally correspond to those described above, and they can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage, such as an electrical, magnetic, or optical memory including a RAM, cache, hard drive, or removable storage disk. Communication device 260 can include any suitable device capable of transmitting and receiving signals over a network, such as a network interface chip or device. The components of the computer can be connected in any suitable manner, such as via a physical bus 270 or wirelessly.

Software 250, which can be stored in memory/storage 240 and executed by processor 210, can include, for example, the programming that embodies the functionality of the present disclosure (e.g., as embodied in the methods and systems described above). Software 250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 240, that can contain or store programming for use by or in connection with an instruction execution system, apparatus, or device.

Software 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can implement any operating system suitable for operating on the network. Software 250 can be written in any suitable programming language, such as C, C++, Java, or Python. In various implementations, application software embodying the functionality of the present disclosure can be deployed in different configurations, such as in a client/server arrangement or through a web browser as a web-based application or web service, for example.

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Sequencing Using Diphosphonic Acid Deprotection of Reversible Terminators In Situ

This example provides a workflow for performing a nucleic acid sequencing reaction comprising diphosphonic acid deprotection of 3′-ONH₂ blocked priming strands to sequence a template nucleic acid molecule (e.g., DNA or RNA) in a tissue section. Use of diphosphonic acid for in situ sequencing may provide certain advantages such as by minimizing tissue damage that occurs during deprotection of 3′-ONH₂ blocked priming strands.

A tissue sample is obtained and cryosectioned onto a glass slide for processing. The tissue is fixed by incubating in 3.7% paraformaldehyde (PFA). In other cases, a FFPE sample can be de-paraffinized and processed to be used. One or more washes are performed and the tissue is then permeabilized. To prepare for probe hybridization, a wash buffer is added to the tissue sample. The washed tissue sample is then contacted with a circularizable probe comprising a template nucleic acid molecule and a barcode sequence. The barcode sequence identifies a target analyte within the tissue sample. The circularizable probe that includes a primer binding site is allowed to hybridize to a “left-hand” portion and a “right-hand” portion of the target analyte. A 20-nucleotide gap between the left-hand and right hand portions of the circularizable probe is filled by a polymerase gapfill reaction. Next, the tissue sample is contacted with a ligation reaction mix including ligase, and the circularizable probe is ligated to form a circular template for rolling circle amplification (RCA). The tissue sample is then incubated with an RCA mixture containing a primer, a strand displacing DNA polymerase, and dNTP for RCA of the circularized probes. From this amplification, the RCA product (e.g., RCP) comprises the template nucleic acid molecule and a barcode sequence.

The tissue sample is washed and then contacted with a sequencing primer. The sequencing primer is allowed to hybridize to the template nucleic acid in the RCP.

A first sequencing cycle is performed as follows. The tissue sample is contacted with a polymerase and fluorescently labelled 3′-ONH₂ blocked nucleotides, allowing a blocked nucleotide to incorporate into the priming strand. The tissue sample is then washed to remove polymerase and unbound nucleotides. Fluorescence imaging is used to detect a signal (or absence of signal) associated with a particular blocked nucleotide incorporated into the priming strand and to identify a complementary nucleotide in the template nucleic acid molecule. Images for each of a plurality of detection channels configured to detect signals from fluorescently labeled nucleotides present in the priming strand are acquired during the sequencing cycle.

Next, to de-block the blocked nucleotide incorporated into the priming strand, the sample is contacted with a reagent including a tetrasodium salt of a diphosphonic acid of Formula (P1):

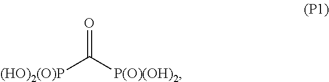

at a concentration of between 10 mM and 50 mM, thereby generating a free 3′-OH in the priming strand in the cell or tissue sample.

The sequencing cycle is repeated in multiple rounds to obtain a sequence of the target analyte.

### Example 2: Tissue Compatibility of Diphosphonic Acid During In Situ Imaging

This example demonstrates the tissue compatibility of a tetrasodium salt of a diphosphonic acid of Formula (P2). Mouse brain tissue sections were prepared on a slide for in situ gene expression detection. Briefly, the tissue was permeabilized and then hybridized with circularizable probes targeting genes known to be expressed in mouse brain tissue, the probes were ligated to form circularized probes, and rolling circle amplification was performed. Next a sequencing by hybridization reaction was performed using fluorescent probes that bind to rolling circle amplification products derived from the targeted genes. Multiple cycles of exposure to fluorescent detection probes followed by stripping of the probes were performed, and each cycle included a step of treatment with the tetrasodium salt of the diphosphonic acid of Formula (P2) at various concentrations and pH. Images were taken after 30 cycles, 60 cycles, and 90 cycles. The conditions tested are shown in Table. 1.

TABLE 1

| Reagent | Concentration | pH |
|---|---|---|
| PBS (negative control) | 1X | 7.4 |
| Formula (P2) | 20 mM | 5.5 |
| Formula (P2) | 60 mM | 5.5 |
| Formula (P2) | 20 mM | 7.4 |
| Formula (P2) | 60 mM | 7.4 |

Results indicated that, for both concentrations at pH 7.4, and for the 20 mM concentration at pH 5.5, the detected RCP intensity during imaging remained stable for 90 cycles of exposure to the diphosphonic acid salt. In fact, for the two concentrations at pH 7.4, the RCP intensity during imaging was comparable to (at least about 80% of) the intensity seen for the PBS control, in all images (at 30 cycles, 60 cycles, and 90 cycles). However, at the 60 mM concentration at pH 5.5, the RCPs remained detectable, but the intensity was dramatically lower than for the other conditions tested. These results demonstrate tissue compatibility of the diphosphonic acid salt during in situ imaging.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

What is claimed is:

1. A method of generating a nucleic acid strand comprising a free 3′-OH in a cell or tissue sample comprising reacting a blocked nucleic acid strand comprising a 3′-O—NH₂ at a 3′ terminus with a diphosphonic acid of Formula (P1) or (P2): 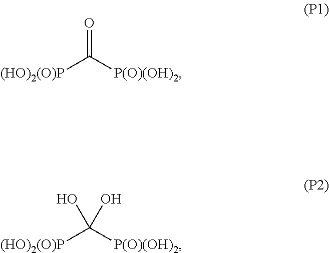
or a salt of Formula (P1) or (P2).

2. The method of claim 1, wherein the diphosphonic acid of Formula (P1) or (P2) or salt of Formula (P1) or (P2) comprises a sodium salt of (P1) or (P2).

3. The method of claim 2, wherein the sodium salt of (P1) or (P2) comprises a mono-, di-, tri-, or tetra-sodium salt of (P1) or (P2).

4. A method of de-blocking a 3′ terminal nucleotide of a priming strand of a sequencing reaction, comprising:
(a) providing a cell or tissue sample comprising: a priming strand bound to a template nucleic acid molecule, wherein the priming strand comprises a blocked nucleotide comprising a 3′-O—NH₂ at a 3′ terminus; and
b) reacting the —O—NH₂ of the blocked nucleotide with a diphosphonic acid of Formula (P1) or (P2): 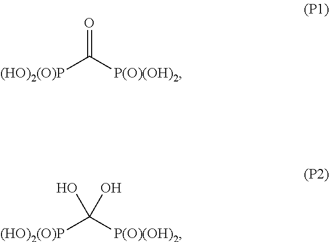
or a salt of Formula (P1) or (P2),
thereby generating a free 3′-OH at the 3′ terminus of the priming strand in the cell or tissue sample.

5. The method of claim 4, wherein the blocked nucleotide is of Formula (I): 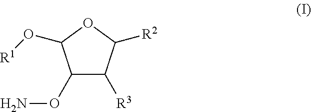
wherein R¹ is H, a monophosphate, a diphosphate, a triphosphate or a nucleic acid; R² is a nucleobase or a modified nucleobase; and R³ is H or OH.

6. The method of claim 5, wherein R² is a modified nucleobase comprising a fluorescent group.

7. The method of claim 4, wherein the diphosphonic acid of Formula (P1) or (P2) or salt of Formula (P1) or (P2) comprises a sodium salt of (P1) or (P2).

8. The method of claim 7, wherein the sodium salt of (P1) or (P2) comprises a mono-, di-, tri-, or tetra-sodium salt of (P1) or (P2).

9. A method of sequencing at least a portion of a template nucleic acid in a cell or tissue sample, the method comprising:
(a) performing a cycle of a nucleic acid sequencing reaction, comprising:
using a polymerase, incorporating a blocked nucleotide comprising a 3′-O—NH₂ into a priming strand bound to the template nucleic acid, and
identifying a nucleotide in the template nucleic acid; and
(b) reacting the blocked nucleotide incorporated into the priming strand with a diphosphonic acid of Formula (P1) or (P2): 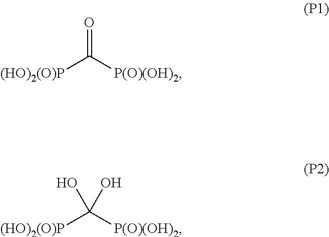
or a salt of Formula (P1) or (P2),
thereby generating a free 3′-OH in the priming strand in the cell or tissue sample.

10. The method of claim 9, wherein the blocked nucleotide is of Formula (I): 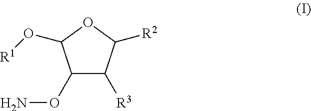
wherein R¹ is H, a monophosphate, a diphosphate, a triphosphate or a nucleic acid; R² is a nucleobase or a modified nucleobase; and R³ is H or OH.

11. The method of claim 10, wherein R² is a modified nucleobase comprising a fluorescent group.

12. The method of claim 11, wherein the method further comprises: cleaving the fluorescent group from the blocked nucleotide after (a).

13. The method of claim 9, wherein performing the cycle of the sequencing reaction comprises detecting the blocked nucleotide incorporated into the priming strand, thereby identifying a nucleotide at a complementary position in the template nucleic acid.

14. The method of claim 9, wherein performing the cycle of the sequencing reaction comprises contacting the priming strand bound to the template with a polymerase and a free nucleotide molecule to generate a ternary polymerase complex, and detecting the ternary polymerase complex.

15. The method of claim 9, wherein the diphosphonic acid of Formula (P1) or (P2) or salt of Formula (P1) or (P2) comprises a sodium salt of (P1) or (P2).

16. The method of claim 15, wherein the sodium salt of (P1) or (P2) comprises a mono-, di-, tri-, or tetra-sodium salt of (P1) or (P2).

17. The method of claim 9, comprising performing at least one additional cycle of the nucleic acid sequencing reaction to identify at least one additional base of the template nucleic acid, the at least one additional cycle comprises:
using a polymerase, incorporating an additional blocked nucleotide comprising a 3′-O—NH₂ into the priming strand bound to the template nucleic acid to generate an extended priming strand; and
identifying a nucleotide in the template nucleic acid.

18. The method of claim 17, wherein the at least one additional cycle comprises reacting the additional blocked nucleotide incorporated into the extended priming strand with a diphosphonic acid of Formula (P1) or (P2): 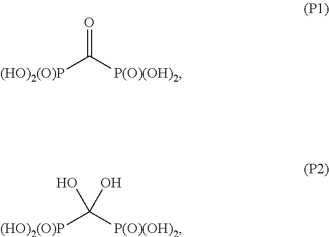
or a salt of Formula (P1) or (P2),
thereby generating a free 3′-OH in the extended priming strand in the cell or tissue sample.

19. The method of claim 17, wherein the at least one additional cycle comprises at least 2, 5, 10, 20, 30, 40, or 50 additional cycles, wherein the additional cycles individually and independently comprise incorporating an additional blocked nucleotide to generate an extended priming strand into the priming strand bound to the template nucleic acid; and
identifying a nucleotide in the template nucleic acid.

20. The method of claim 9, wherein the nucleic acid sequencing reaction is performed at a pH between pH 6 and pH 10.

21. The method of claim 9, comprising contacting the cell or tissue sample with a mixture of 3′-O—NH₂ blocked nucleotides comprising at least two different nucleobases selected from the group consisting of A, T or U, C, and G.

22. The method of claim 9, wherein the template nucleic acid comprises a target analyte nucleic acid sequence.

23. The method of claim 9, wherein the template nucleic acid comprises a barcode sequence associated with an analyte in the cell or tissue sample.

24. The method of claim 9, wherein the cell or tissue sample is immobilized on a surface.

25. The method of claim 9, wherein the reacting the —O—NH₂ of the blocked nucleotide with the diphosphonic acid of Formula (P1) or (P2) or the salt thereof comprises contacting the cell or tissue sample with a reagent comprising the diphosphonic acid of Formula (P1) or (P2) or the salt thereof at a concentration in a range of 1 mM to 100 mM.
